(19) United States
(12) Patent Application Publication
(10) Pub. No.: US 2026/0232182 A1
(43) Pub. Date: Aug. 13, 2026

(54) Systems and Methods for Performing Vision Assessments

(71) Applicant: Radius XR, LLC, Pleasanton, CA (US)

(72) Inventors: Chris Bradley, Baltimore, MD (US); John Trefethen, Bellingham, WA (US)

(21) Appl. No.: 19/461,512

(22) Filed: Jan. 27, 2026

Related U.S. Application Data
(60) Provisional application No. 63/751,186, filed on Jan. 29, 2025.

(51) Int. Cl. A61B 3/024; A61B 3/00

(52) U.S. Cl. CPC A61B 3/024; A61B 3/005

(57) ABSTRACT

Systems and methods for performing pointwise progression analysis include generating visual field test data by conducting a series of visual field eye exams of a patient over a period of time. Visual field test data, at multiple test locations of the patient's eyes, is analyzed in order to generate a graphical user interface and display a rate of change of sensitivity at each of the test locations and highlight a rate of change if the results are further determined to be significant.

### CROSS-REFERENCE

The present application relies on United States Patent Provisional Application No. 63/751,186, titled “Systems and Methods for Performing and Displaying Pointwise Progression Analysis Based on Eye Exam Test Data” and filed on Jan. 29, 2025, for priority, which is herein incorporated by reference in its entirety.

### FIELD

The present specification relates to vision assist and/or diagnostic systems and methods. Specifically, the embodiments disclosed herein describe systems and methods for generating a pointwise progression analysis based on test data corresponding to a series of eye exams of a patient conducted over a period of time.

### BACKGROUND

Visual field progression provides clinical signs of deterioration in optic neuropathies such as glaucoma. There are conventionally two broad classes of visual field progression analyses: trend-based analysis and event-based analysis. Trend-based analysis typically involves linear regression techniques (i.e. fitting a line through test data corresponding to a patient's eye exams conducted over a period of time) in order to generate a plurality of statistical analyses, such as the rate of change in mean deviation, pattern standard deviation and visual field index over time. Event-based analysis involves looking at a plurality of visual field tests (for example, five visual field tests) to determine whether there is a significant change between baseline (first two visual fields) and post-baseline (last three visual fields).

GPA (Guided Progression Analysis) is a type of event-based analysis that produces a “likely progression” alert (also referred to as an “event”) when the same three or more test points are flagged as having changed compared to baseline in three or more eye exams or tests in a row. In three-point GPA, an “event” is defined as three or more points showing significant change from baseline to post-baseline, while a five-point GPA refers to the same analysis, but using five points.

The five points that show significant change in the five-point GPA, can be anywhere in the visual field and does not take into account biological plausibility. Also, the normative distributions used to calculate statistical significance are not readily available, which would lead to variance between different statistical tests.

Accordingly, there is a need for systems and methods for conducting a pointwise progression analysis that integrates both trend-based and event-based analysis. There is also a need for systems and methods that generate at least one graphical user interface to display and highlight data indicative of significant trend-based or event-based changes at each test location of the visual field.

### SUMMARY

The following embodiments and aspects thereof are described and illustrated in conjunction with systems, tools and methods, which are meant to be exemplary and illustrative, and not limiting in scope. The present application discloses numerous embodiments.

In some embodiments, the present specification discloses a computer-implemented method for performing an assessment of a patient's vision, comprising: providing a head-mounted vision device configured to be positioned on the patient's head, wherein the head-mounted vision device comprises a display; providing a first computing device in data communication with the head-mounted vision device; providing at least one server in data communication with at least one of the first computing device and the head-mounted vision device; providing a non-transient computer readable medium that is in data communication with at least one of the at least one server and the first computing device, wherein the non-transient computer readable medium comprises a plurality of programmatic instructions that, when executed by at least one processor in at least one of the first computing device and the at least one server: presenting a series of visual field tests to the patient over a period of time, wherein each of the visual field tests is defined by a visual field test pattern having a plurality of test locations in a visual field of the patient; receiving visual field test data for each of location of the plurality of test locations; applying a function to the visual field test data in order to determine a functional relationship between visual field test data at a given one of the plurality of test locations across the series of visual field tests conducted over the period of time; for each of the plurality of test locations, using the functional relationship to determine a value indicative of a change of sensitivity in the patient's visual field at each of the plurality of test locations; permuting the series of visual field test data at some or all of the plurality of test locations to generate a unique number of permuted sets of test data; using the permuted sets of test data, generating a plurality of additional values for each of the plurality of test locations; generating a distribution of the plurality of additional values for each of the plurality of test locations; and determining if the change of sensitivity at a given one of the plurality of test locations is significant based on the distribution.

Optionally, the function applied to the series of test data is a linear regression function.

Optionally, the functional relationship between visual field test data at said given one of the plurality of test locations is defined by a linear regression line.

Optionally, the method further comprises obtaining said linear regression line for every one of the plurality of test locations across said series of test data.

Optionally, using the functional relationship to determine the value indicative of the change of sensitivity in the patient's visual field comprises calculating a slope of the linear regression line for each of the plurality of test locations, wherein the slope is indicative of a rate of change of sensitivity at each of the plurality of test locations.

Optionally, using the permuted sets of test data to generate the plurality of additional values comprises generating a plurality of slope values for each of the plurality of test locations.

Optionally, said generation of the plurality of slope values for each of the plurality of test locations comprises fitting a line through each permuted test data by applying linear regression to the permuted test data and calculating a slope of the linear regression line for either the unique number of permuted test data or for a predetermined number of randomly chosen permutations from the unique number of permuted test data.

Optionally, the visual field test data are indicative of a degree of sensitivity at a given one of the plurality of test locations in the patient's visual field.

Optionally, the series of visual field test data includes N data, and wherein the number of permuted sets of test data is N! (N factorial).

Optionally, the distribution is indicative of an effect of random noise on the series of visual field test data at a given one of the plurality of test locations.

Optionally, said significance of the change of sensitivity at the given one of the plurality of test locations is determined based on whether both a first condition and a second condition are true.

Optionally, the first condition is indicative of whether the change of sensitivity at the given one of the plurality of test locations is worse than a first predefined threshold.

Optionally, the first predefined threshold is within a range of −0.3 dB/year to −1.5 dB/year.

Optionally, the second condition is indicative of whether the change of sensitivity at the given one of the plurality of test locations is lower than a second predefined threshold of the distribution of the plurality of additional values for each of the plurality of test locations.

Optionally, the plurality of additional values comprises a plurality of slope values for each of the plurality of test locations.

Optionally, the method further comprises causing at least one graphical user interface to be generated in the display of the head-mounted vision device or in the first computing device, wherein the graphical user interface is configured to display the change of sensitivity at a given one of the plurality of the test locations.

Optionally, the graphical user interface is further configured to visually highlight whether the change of sensitivity at the given one of the plurality of the test locations is significant.

Optionally, the graphical user interface is further configured to display a message indicative of a total number of the plurality of the test locations determined to have significant changes.

Optionally, the change of sensitivity is a rate of change of sensitivity.

In some other embodiments, the present specification discloses a computer-implemented method of generating a pointwise progression analysis at each of a plurality of test locations of a patient's eye, comprising: receiving a series of test data for each of the plurality of test locations, wherein the series of test data is recorded by performing a series of visual field tests over a period of time; applying linear regression to the series of test data in order to generate a linear regression line for each of the plurality of test locations; calculating a slope of the linear regression line for each of the plurality of test locations, wherein the slope is indicative of a rate of change of sensitivity at each of the plurality of test locations; permuting the series of test data at a test location to generate a unique number of permuted sets of test data; generating a plurality of slope values for the test location by performing the following steps for either the unique number of permuted test data or for a predetermined number of randomly chosen permutations from the unique number of permuted test data: fitting a line through each permuted test data by applying linear regression to the permuted test data and calculating the slope of the linear regression line; generating a distribution of the plurality of slope values for the test location; and determining if the rate of change of sensitivity at the test location is significant.

Optionally, the test data corresponds to a sensitivity estimate.

Optionally, the rate of change is in dB/year.

Optionally, the series of test data includes N data, and wherein the number of permuted test data is N! (factorial N).

Optionally, the distribution is indicative of an effect of random noise on the series of test data at the test location.

Optionally, significance of the rate of change of sensitivity at the test location is determined based on whether a first condition as well as a second condition are true. Optionally, the first condition determines if the rate of change of sensitivity at the test location is worse than a first predefined threshold. Optionally, the first predefined threshold is −0.9 dB/year.

Optionally, the second condition determines if the rate of change of sensitivity at the test location is lower than a second predefined threshold of the distribution of the plurality of slope values for the test location.

Optionally, the computer-implemented method of further comprises: generating at least one graphical user interface for displaying the rate of change of sensitivity at the test location, b) highlighting the test location if the rate of change of sensitivity at the test location is determined to be significant, and displaying a textual message indicative of the total number of test locations determined to have significant rate of changes.

The present specification also discloses a computer readable program adapted to deliver an eye exam order to a patient using a head-mounted vision device positioned on the patient's head, wherein the head-mounted vision device includes a display in electrical and data communication with a first computing device that is in data communication with at least one server over a network, wherein the first computing device includes at least one processor in data communication with a non-transient memory that stores the computer readable program, and wherein the computer readable program comprises a plurality of programmatic instructions that, when executed by the at least one processor: receives a series of test data for each of the plurality of test locations, wherein the series of test data is recorded by performing a series of visual field tests over a period of time; applies linear regression to the series of test data in order to generate a linear regression line for each of the plurality of test locations; calculates a slope of the linear regression line for each of the plurality of test locations, wherein the slope is indicative of a rate of change of sensitivity at each of the plurality of test locations; permutes the series of test data at a test location to generate a unique number of permuted test data; generates a plurality of slope values for the test location by performing the following steps for either the unique number of permuted test data or for a predetermined number of randomly chosen permutations from the unique number of permuted test data: fitting a line through each permuted test data by applying linear regression to the permuted test data and calculating the slope of the linear regression line; generates a distribution of the plurality of slope values for the test location; and determines if the rate of change of sensitivity at the test location is significant.

Optionally, the test data corresponds to a sensitivity estimate.

Optionally, the rate of change is in dB/year.

Optionally, the series of test data includes N data, and wherein the number of permuted test data is N! (factorial N).

Optionally, the distribution is indicative of an effect of random noise on the series of test data at the test location.

Optionally, the significance of the rate of change of sensitivity at the test location is determined based on whether a first condition as well as a second condition are true. Optionally, the first condition determines if the rate of change of sensitivity at the test location is worse than a first predefined threshold. Optionally, the first predefined threshold is −0.9 dB/year.

Optionally, the second condition determines if the rate of change of sensitivity at the test location is lower than a second predefined threshold of the distribution of the plurality of slope values for the test location.

Optionally, the plurality of programmatic instructions, when executed by the at least one processor: generates at least one graphical user interface for displaying the rate of change of sensitivity at the test location, b) highlights the test location if the rate of change of sensitivity at the test location is determined to be significant, and displays a textual message indicative of the total number of test locations determined to have significant rate of changes.

The present specification also discloses a method of generating an analysis at each of a plurality of test locations of a patient's eye, the method comprising: receiving a series of test data for each of the plurality of test locations, wherein the series of test data is recorded by performing a series of visual field tests over a period of time; applying linear regression to the series of test data in order to generate a linear regression line for each of the plurality of test locations; calculating a slope of the linear regression line for each of the plurality of test locations, wherein the slope is indicative of a rate of change of sensitivity at each of the plurality of test locations; permuting the series of test data at a test location to generate a unique number of permuted test data; generating a plurality of slope values for the test location by performing the following steps for either the unique number of permuted test data or for a predetermined number of randomly chosen permutations from the unique number of permuted test data: fitting a line through each permuted test data by applying linear regression to the permuted test data and calculating the slope of the linear regression line; generating a distribution of the plurality of slope values for the test location; and determining if the rate of change of sensitivity at the test location is significant, wherein the significance of the rate of change of sensitivity at the test location is determined based on whether a first condition as well as a second condition are true, wherein the first condition determines if the rate of change of sensitivity at the test location is worse than a first predefined threshold, and wherein the second condition determines if the rate of change of sensitivity at the test location is lower than a second predefined threshold of the distribution of the plurality of slope values for the test location.

Optionally, the method further comprises: generating at least one graphical user interface for displaying the rate of change of sensitivity at the test location, b) highlighting the test location if the rate of change of sensitivity at the test location is determined to be significant, and displaying a textual message indicative of the total number of test locations determined to have significant rate of changes.

The aforementioned and other embodiments of the present specification shall be described in greater depth in the drawings and detailed description provided below.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries (e.g. boxes, groups of boxes, or other shapes) in the figures represent one example of the boundaries. It may be that in some examples one element may be designed as multiple elements or that multiple elements may be designed as one element. In some examples, an element shown as an internal component of one element may be implemented as an external component in another and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1A is a flowchart describing steps of a method for generating a trend-based analysis using test data corresponding to a series of eye exams of a patient, in accordance with some embodiments of the present specification;

FIG. 1B is a flowchart describing steps of a method for generating an event-based analysis using the test data corresponding to the series of eye exams of the patient, in accordance with some embodiments of the present specification;

FIG. 2 is a GUI (Graphical User Interface) generated by the VAD App to display data indicative of a pointwise progression analysis (PPA), in accordance with some embodiments of the present specification;

FIG. 3 is a block diagram showing a clinical setup of a vision assist and/or diagnostic system, in accordance with some embodiments of the present specification;

FIG. 4 is a flowchart of a plurality of exemplary steps of a method of delivering an eye exam order to a patient, in accordance with some embodiments of the present specification;

FIG. 5A is a first GUI generated by a VAD App, in accordance with some embodiments of the present specification;

FIG. 5B is a second GUI generated by the VAD App, in accordance with some embodiments of the present specification;

FIG. 5C represent first and second views of a third GUI generated by the VAD App, in accordance with some embodiments of the present specification;

FIG. 5D represents first, second, third, fourth and fifth views of a fourth GUI generated by the VAD App, in accordance with some embodiments of the present specification;

FIG. 5E is a fifth GUI generated by the VAD App, in accordance with some embodiments of the present specification;

FIG. 5F represents first and second views of a sixth GUI generated by the VAD App, in accordance with some embodiments of the present specification; and

FIG. 6 is a GUI generated by the VAD App which is configured to enable creation of a custom eye exam order for a patient, in accordance with some embodiments of the present specification.

### DETAILED DESCRIPTION

The present specification is directed towards multiple embodiments. The following disclosure is provided in order to enable a person having ordinary skill in the art to practice the invention. Language used in this specification should not be interpreted as a general disavowal of any one specific embodiment or used to limit the claims beyond the meaning of the terms used therein. The general principles defined herein may be applied to other embodiments and applications without departing from the spirit and scope of the invention. Also, the terminology and phraseology used is for the purpose of describing exemplary embodiments and should not be considered limiting. Thus, the present invention is to be accorded the widest scope encompassing numerous alternatives, modifications and equivalents consistent with the principles and features disclosed. For purpose of clarity, details relating to technical material that is known in the technical fields related to the invention have not been described in detail so as not to unnecessarily obscure the present invention.

In various embodiments, a computing device includes an input/output controller, at least one communications interface and system memory. The system memory includes at least one random access memory (RAM) and at least one read-only memory (ROM). These elements are in communication with a central processing unit (CPU) to enable operation of the computing device. In various embodiments, the computing device may be a conventional standalone computer or alternatively, the functions of the computing device may be distributed across multiple computer systems and architectures.

In some embodiments, execution of a plurality of sequences of programmatic instructions or code enable or cause the CPU of the computing device to perform various functions and processes. In alternate embodiments, hard-wired circuitry may be used in place of, or in combination with, software instructions for implementation of the processes of systems and methods described in this application. Thus, the systems and methods described are not limited to any specific combination of hardware and software.

The term “module”, “application” or “engine” used in this disclosure may refer to computer logic utilized to provide a desired functionality, service or operation by programming or controlling a general purpose processor. Stated differently, in some embodiments, a module, application or engine implements a plurality of instructions or programmatic code to cause a general purpose processor to perform one or more functions. In various embodiments, a module, application or engine can be implemented in hardware, firmware, software or any combination thereof. The module, application or engine may be interchangeably used with unit, logic, logical block, component, or circuit, for example. The module, application or engine may be the minimum unit, or part thereof, which performs one or more particular functions.

In the description and claims of the application, each of the words “comprise”, “include”, “have”, “contain”, and forms thereof, are not necessarily limited to members in a list with which the words may be associated. Thus, they are intended to be equivalent in meaning and be open-ended in that an item or items following any one of these words is not meant to be an exhaustive listing of such item or items, or meant to be limited to only the listed item or items. It should be noted herein that any feature or component described in association with a specific embodiment may be used and implemented with any other embodiment unless clearly indicated otherwise.

It must also be noted that as used herein and in the appended claims, the singular forms “a,” “an,” and “the” include plural references unless the context dictates otherwise. Although any systems and methods similar or equivalent to those described herein can be used in the practice or testing of embodiments of the present disclosure, the preferred systems and methods are now described.

As used herein, the term “isopter” refers to a contour of similar visual sensitivity estimates. It is similar to a topographical map where there are contours representing similar elevations. Thus, in technical terms, an isopter is a contour line in a representation of the visual field around the points representing the macula that passes through the points of equal visual acuity.

In embodiments of the present specification, it should be noted that while threshold and sensitivity refer to the same data that is being generated, they are inverses of each other (sensitivity=1/threshold).

### Method(s) for Generating a Pointwise Progression Analysis (PPA)

In accordance with aspects of the present specification, a vision assist and diagnostic application, module, or engine (hereinafter referred to as a “VAD App”) implements a plurality of instructions of programmatic code to configure a computing device to perform analyses of collective test data corresponding to a plurality of eye exams of a patient conducted over a period of time.

In some embodiments, each of the plurality of eye exams is a visual field test. For example, there may be a total of ‘L’ visual field tests/exams performed for a given eye of the patient over time. In embodiments, a visual field exam is used to test K locations at which sensitivity estimates are generated over time. In some embodiments, the number of test locations K corresponds to the number of test locations in a standard visual field test pattern such as, for example, K=54 test locations in a standard 24-2 visual field test, and K=68 locations in a standard 10-2 visual field test. The number of test locations K may vary in the case of custom visual field tests/exams. In an embodiment, the sensitivity estimate is generated based on a “visual threshold” at each location, meaning the brightness or intensity at which the patient will view or observe a stimulus 50% of the time at that location.

In some embodiments, the VAD App is used to configure the computing device to perform a pointwise progression analysis (PPA) based on the test data corresponding to the plurality of eye exams of a patient conducted over a period of time. In accordance with aspects of the present specification, the PPA is a combination of trend-based analysis and event-based analysis.

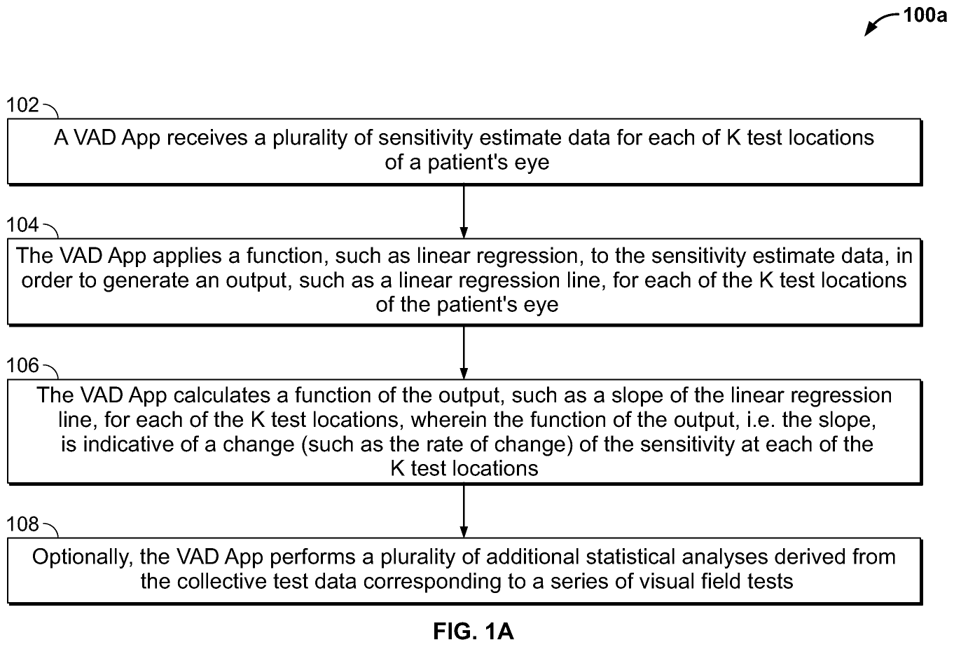

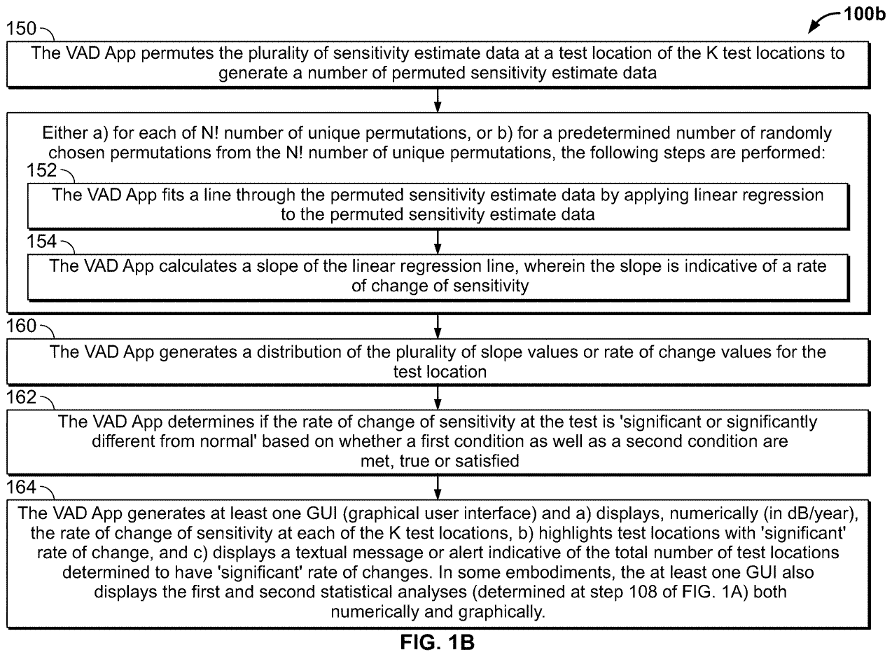

In embodiments, the trend-based portion of the PPA enables a clinician to view the patient's test data trends, over a period of time, at each of the K test locations/points. To generate a trend analysis, the VAD App implements a first method (see method 100a of FIG. 1A) that is configured to apply a linear regression to all sensitivity estimates data (i.e. the test data) at each of the K test locations, to measure a rate of change, expressed in dB/year, at each of the K test locations.

In embodiments, the event-based portion of the PPA is configured to indicate, to the clinician, if the rate of change in dB/year at each of the K test locations/points is ‘significant or significantly different from normal’, as described in detail below. To generate an event-based analysis, the VAD App implements a second method (see method 100b of FIG. 1B) to determine and highlight test locations for which the rate of change is determined to be ‘significant’.

In accordance with some embodiments, in order to perform PPA, the VAD App acquires visual field test data, over time, based on a series of eye tests/exams of the patient. Each eye test examines the same set of points or test locations. In some embodiments, in order to perform PPA, the VAD App requires at least three visual field tests/exams, as input, over time. In contrast, most traditional event-based analysis, including GPA, requires precisely five visual fields and most traditional trend-based analysis requires at least five visual fields but can be performed on three or more visual fields.

FIG. 1A is a flowchart describing steps of a method 100a for generating a trend-based analysis using test data corresponding to a series of eye exams of a patient, in accordance with some embodiments of the present specification. In embodiments, the VAD App is configured to implement method 100a.

Referring to FIG. 1A, at step 102, the VAD App receives a plurality of sensitivity estimate data (also referred to as ‘test data’) for each of K test locations of the patient's eye. The plurality of sensitivity estimate data is recorded by performing a series of visual field tests over a period of time, wherein, for each visual field test, sensitivity estimate data is recorded for each of the same K test locations of the patient's visual field.

At step 104, the VAD App applies a function, such as a linear regression function, to the plurality of sensitivity estimate data, in order to generate an output, such as a linear regression line, for each of the K test locations/points of the patient's eye. In embodiments, each of the plurality of sensitivity estimate data (for each of the K test locations) is associated with a specific time of measurement that corresponds to the time at which the visual field test was performed and the sensitivity estimate data was generated. In some embodiments, a linear regression function is applied to the plurality of sensitivity estimate data arranged in an ascending order of the specific time associated with each of the plurality of sensitivity estimate data.

At step 106, the VAD App calculates a function of the output, such as a slope of the linear regression line, for each of the K test locations, wherein the function of the output is indicative of a change in the sensitivity at each of the K test locations, such as a slope which is indicative of a rate of change of the sensitivity at each of the K test locations. In some embodiments, the rate of change is measured in units of dB/year. Stated differently, the rate of change of the sensitivity is calculated for each test location of the K test locations.

In some embodiments, the function applied to the plurality of sensitivity estimate data may comprise any suitable statistical, mathematical, or computational function configured to characterize a temporal relationship or trend in the sensitivity estimate data across the series of visual field tests. By way of non-limiting example, the function may include polynomial regression, piecewise or segmented regression, robust regression techniques configured to reduce sensitivity to outliers, or non-parametric trend detection functions, including rank-based or distribution-free methods. In some embodiments, the function may comprise a non-linear time-series model, such as an exponential, logarithmic, or state-space model, including Kalman filter-based estimators, configured to account for measurement noise and temporal dynamics. In further embodiments, the function may determine a percentile-based change metric, a standardized change score, or another statistical summary indicative of sensitivity progression over time.

Optionally, at step 108, the VAD App performs a plurality of additional statistical analyses using the collective test data corresponding to the series of visual field tests. In a non-limiting scenario, the plurality of additional statistical analyses include, for example, calculating the rate of change in mean deviation, pattern standard deviation and/or visual field index.

FIG. 1B is a flowchart describing steps of a method 100b for generating an event-based analysis to determine if a rate of change of sensitivity, at each of the K test locations/points of the patient's visual field, is statistically significant, in accordance with some embodiments of the present specification. In embodiments, the VAD App implements the method 100b.

Referring to FIG. 1B, at step 150, the VAD App permutes the plurality of sensitivity estimate data at a test location/point of the K test locations/point to generate a number of permuted sensitivity estimate data. For example, if there are N number of sensitivity estimate data (as a result of performing N visual field tests over the period of time) at a given test location, then there would be N! (‘factorial N’) number of permuted sensitivity data (indicative of unique permutations of the plurality of sensitivity estimate data) for the test location.

Subsequently, either a) for each of the N! number of unique permutations, or b) for a predetermined number of randomly chosen permutations from the N! number of unique permutations, the following steps are performed. In some embodiments, it should be noted that the predetermined number is selected to avoid computational complexity—when N is large, N! can be very large, and therefore, the number is selected to ensure that the computations can be performed.

At step 152, the VAD App fits a line through the permuted sensitivity estimate data by applying linear regression to the permuted sensitivity estimate data.

At step 154, the VAD App calculates a slope of the linear regression line, wherein the slope is indicative of a rate of change of sensitivity.

At the end of performing steps 152 and 154 for either a) each of N! number of unique permutations or b) the predetermined number of randomly chosen permutations (from the N! number of unique permutations), the VAD App generates a plurality of slope values or rate of change values for the test location.

At step 160, the VAD App generates a distribution of the plurality of slope values or rate of change values for the test location. The distribution is theoretically indicative of the effect of random noise on the plurality of sensitivity estimate data at the test location.

At step 162, the VAD App determines if the slope or rate of change of sensitivity at the test location (that is, the slope or rate of change of the linear regression line to the original, non-permuted sensitivity estimates at the test location (of the K test locations)—determined at step 106 of method 100a) is ‘significant or significantly different from normal’ based on whether a first condition and a second condition are met, true or satisfied.

The first condition is directed towards determining if the slope or rate of change at the test location is worse (or more negative) than a first predefined threshold. In some embodiments, the first predefined threshold is within a range of about −0.1 dB/year to about −1.8 dB/year and in some other embodiments, the first predefined threshold is within a range of about −0.3 dB/year to about −1.5 dB/year. In some embodiments, the first predefined threshold is −0.9 dB/year. Thus, if the slope is more negative than −0.9 dB/year, then it is considered as greater visual field worsening.

The second condition is directed towards determining if the rate of change at the test location is less or lower than a second predefined threshold of the distribution of the plurality of slope values or rate of change values for the test location. For example, as an illustration, the second predefined threshold may be the 5^{th} percentile of the distribution. Stated differently, if the rate of change at the test location is less or lower (or more negative) than the 5^{th} percentile of the distribution of slopes then the rate of change at the test location is considered ‘significant’ or ‘statistically significant’and indicates greater visual field deterioration.

It should be appreciated that steps 150, 152, 154, 160 and 162 are repeated for each of the K test locations. thereby determining if the rate of change of sensitivity at each of the K test locations is ‘significant’.

Thus, linear regression is applied to each permuted set of sensitivity estimates at a test location, and the distribution of slopes across all permuted sets of sensitivity estimates, at the test location, is used to determine whether a measured slope (the slope of the linear regression line to the original, non-permuted sensitivity estimates) at the test location is statistically significant, for example, by assessing whether the measured slope lies below the 5th percentile of the distribution of slopes.

At step 164, the VAD App generates at least one GUI (graphical user interface) and a) displays, numerically (in dB/year), the rate of change of sensitivity at each of the K test locations, b) highlights test locations with ‘significant’ rate of change, and c) displays a textual message or alert indicative of the total number of test locations determined to have ‘significant’ rate of changes. In some embodiments, the at least one GUI also displays the plurality of additional statistical analyses (determined at step 108) both numerically and graphically. In embodiments, the plurality of additional statistical analyses includes a first statistical analysis and a second statistical analysis.

Thus, the VAD App performs a (pointwise) trend-based analysis at each test location (of K test locations) in order to generate and display, in a GUI, a rate of change of sensitivity at each test location. The VAD App also determines and highlights, in the GUI, which test locations (of the K test locations) have rates of change that are ‘significant or significantly different from normal’. The number of test locations with ‘significant’rates of change constitute an ‘event’.

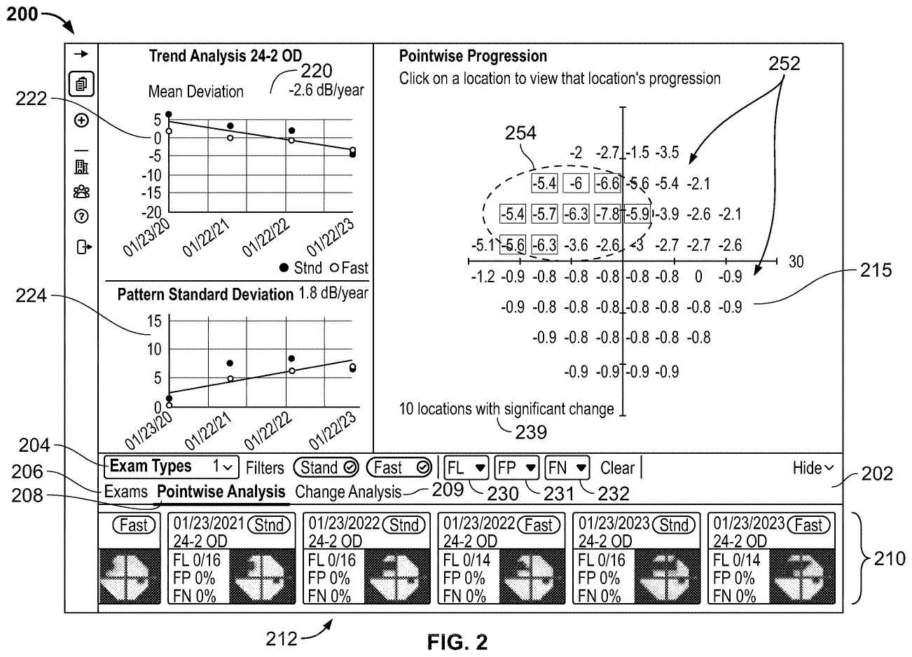

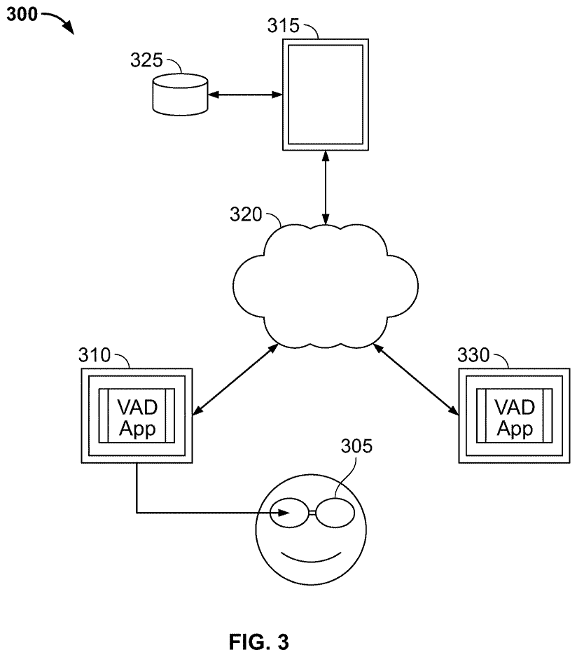

FIG. 2 is a GUI 200 generated by the VAD App to display data indicative of the pointwise progression analysis (PPA), in accordance with some embodiments of the present specification. The GUI 200 is displayed on a computing device, such as, for example, the first computing device 310 and second computing device 330 in the exemplary clinical setup of FIG. 3.

In some embodiments, the GUI 200 is configured such that it enables selection of one or more of a plurality of eye exam types provided to the patient at various dates and times. As shown in FIG. 2, the GUI 200 includes a bottom portion 202 having a drop-down box 204 configured to enable filtering of a plurality of eye exams on the basis of an eye exam type. Positioned below the drop-down box 204 are a first visual graphical element 206, a second visual graphical element 208 and a third visual graphical element 209. The first visual graphical element 206, when actuated, is configured to enable visualization of one or more eye exams based on an eye exam type selected from the drop-down box 204 and related data, functionalities and features. The second visual graphical element 208, when actuated, is configured to display and enable data, functionalities and features related to PPA (Pointwise Progression Analysis) with reference to at least three eye exams of the patient (while, traditional trend-based as well as event-based analysis require at least five visual fields including the event-based analysis, GPA, that requires five visual fields.). The third visual graphical element 209, when actuated, is configured to enable data, functionalities and features related to comparative or change analysis with reference to at least two eye exams of the patient.

Since the second visual graphical element 206 is shown as actuated in the figure, the GUI 200 is configured for displaying and enabling data, functionalities, and features related to PPA (with reference to an eye exam type, such as, for example, a visual field test, selected from the drop-down box 204).

A horizontal tray 210 is configured to display a plurality of eye exams 212, in visual miniature forms/icons, characterized by the eye exam type selected from the drop-down box 204. In some embodiments, by default, the plurality of eye exams 212 are presented left to right sorted in an ascending order of oldest eye exam to newest eye exam. However, the plurality of eye exams 212 may alternatively be sorted in descending order (or any other order) to create the horizontal tray 210.

In embodiments, a fourth visual graphical element 230, a fifth visual graphical element 231, and a sixth visual graphical element 232 are configured as drop-down boxes to enable the plurality of eye exams 212 to be filtered for metrics such as, for example, fixation loss, false positives, and false negatives, respectively. The metrics of fixation loss, false positives and false negatives are indicative of reliability of an eye exam. Consequently, the fourth visual graphical element 230, fifth visual graphical element 231, and sixth visual graphical element 232 may be manipulated to filter out unreliable eye exams. For example, the fourth visual graphical element 230 may be manipulated to filter out eye exams with fixation loss of higher than, say for example, 30%.

A first window 215 displays a PPA data 252 (on a graphical representation of the patient's visual field) at each of a plurality of test locations corresponding to the patient's visual field. The PPA data 252 comprises a numerical value, expressed in dB/year, indicative of a rate of change of sensitivity, over a period of time, at each of the plurality of test locations. It should be appreciated that the numerical value is generated as a result of the VAD App implementing the method 100a of FIG. 1A.

As shown, a subset PPA data 254 of the PPA data 252 is highlighted, in a predefined color, indicating that the highlighted subset PPA data 254 is ‘significant’. In embodiments, the highlighted subset PPA data 254 also enables a clinician to know which of the plurality of test locations show ‘significant’ rates of change. Additionally, a textual message or alert 239 indicates a total number of test locations that have ‘significant’ rates of change (corresponding to the highlighted subset PPA data 254). It should be appreciated that whether a PPA data 252 is ‘significant’ (and, therefore, should be highlighted) is determined as a result of the VAD App implementing the method 100b of FIG. 1B.

The displayed PPA data 252, highlighted subset PPA data 254 and textual message or alert 239 are based on test data corresponding to the plurality of eye exams 212 displayed in the horizontal tray 210. In some embodiments, the pointwise progressive analysis is generated based on at least a predefined number of visual field tests/exams of the patient's eye conducted over a period of time. In some embodiments, the first predetermined number is three.

Also, a plurality of statistical data analyses is displayed in a second window 220, concurrently with the PPA data 252. The first window 215 and second window 220 are positioned adjacent to each other and above the horizontal tray 210. In some embodiments, the plurality of statistical data analysis is derived from the collective test data corresponding to the plurality of eye exams 212 displayed in the horizontal tray 210. In some embodiments, the plurality of statistical data analysis is shown both numerically and graphically and includes at least a first statistical analysis 222 indicative of mean deviation, and a second statistical analysis 224 indicative of pattern standard deviation.

### Exemplary Use Case

In some embodiments, the VAD App is implemented, on one or more computing devices, in a clinical environment or setup configured to generate test data for a plurality of test locations or visual fields corresponding to a series of eye exams of a patient conducted over a period of time.

FIG. 3 is a block diagram showing a clinical setup of a vision assist and/or diagnostic system 300, in accordance with some embodiments of the present specification. The system 300 includes a head-mounted vision device 305 which, when worn or mounted on the head of a patient, is in electrical and data communication with at least one co-located first computing device 310. In some embodiments, the at least one first computing device 310 is in data communication with at least one server 315 over a network 320 that may be wired and/or wireless. In some embodiments, at least one server 315 includes or is in communication with a database system 325. The database system 325 stores a plurality of data corresponding to patients, eye exam orders, eye exams and results of the eye exams. In some embodiments, the at least one server 315 may be implemented by a cloud of computing platforms operating together as the at least one server 315.

In some embodiments, the at least one second computing device 330, is also in data communication either directly with the at least one first computing device 310 and/or is in data communication with the at least one first computing device 310 through the network 320. In some embodiments, the at least one second computing device 330 may be located remotely from the at least one first computing device 310 and the head-mounted vision device 305. Alternatively, the at least one second computing device 330 may be co-located with the at least one first computing device 310 and the head-mounted vision device 305.

In some embodiments, the at least one first computing device 310 is provided with programming through the VAD App. The VAD App implements a plurality of instructions of programmatic code to configure the at least one first computing device 310 to function: a) as a patient check-in device, b) as a technician or clinical staff person's device for inputting or adding eye exam orders, inputting or adding patient information, inputting or adding eye exam order templates and inputting or adding educational content, c) as a patient testing or diagnostic device, and d) as a device for generating and displaying results of various analyses, including at least a pointwise progression analysis (PPA), based on test data corresponding to a plurality of eye exams of a patient conducted over a period of time. The GUI 200 of FIG. 2, as described earlier in this specification, is an exemplary graphical user interface configured to display results of the PPA.

When the at least one first computing device 310 is connected (that is, in electrical and data communication) to the head-mounted vision device 305, the at least one computing device 310 enters into a patient testing mode and drives the head-mounted vision device 305 to deliver an eye exam order comprising a plurality of predefined sequential series of content or agenda.

In some embodiments, the at least one second computing device 330 may also implement the VAD App which may be used by the clinician to access results and various analyses of the patient's eye exams. In some embodiments, the VAD App may be implemented on the at least one server 315 and provided as a service to the at least one first computing device 310 and/or the at least one second computing device 330.

In some embodiments, the head-mounted vision device 305 is a passive display device and does not have its own processor or power source. In some embodiments, the head-mounted vision device 305 has at least one screen (or alternatively, left and right screens corresponding to a patient's left and right eyes) that displays, to the patient, the plurality of predefined sequential series of content or agenda associated with the eye exam order.

In various embodiments, the at least one first computing device 310 and the at least one second computing device 330 comprise devices such as, but not limited to, personal or desktop computers, laptops, Netbooks, handheld devices such as smartphones, tablets, and PDAs and/or any other computing platform known to persons of ordinary skill in the art.

### Eye Exam Order Delivery Method

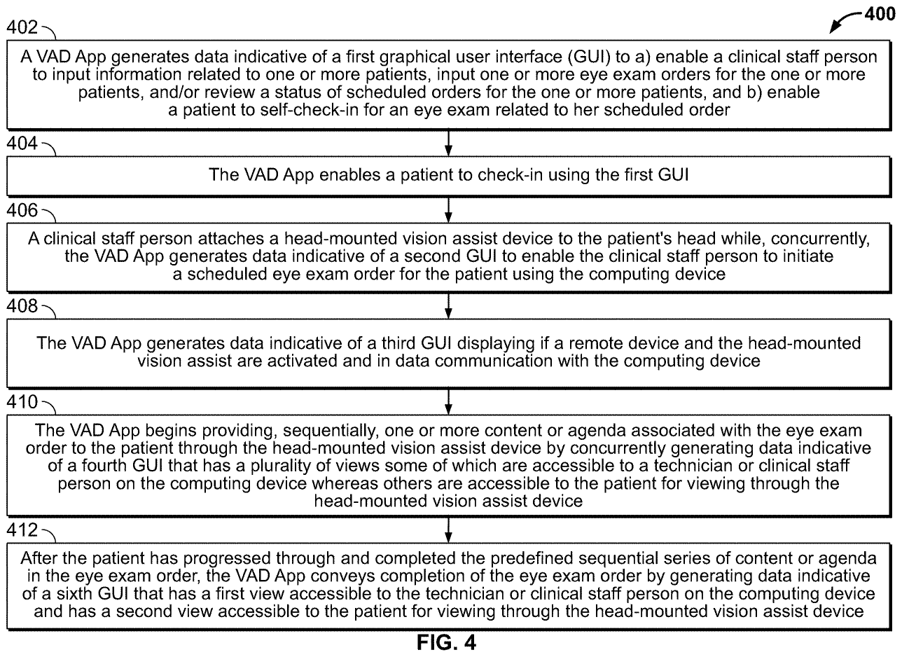

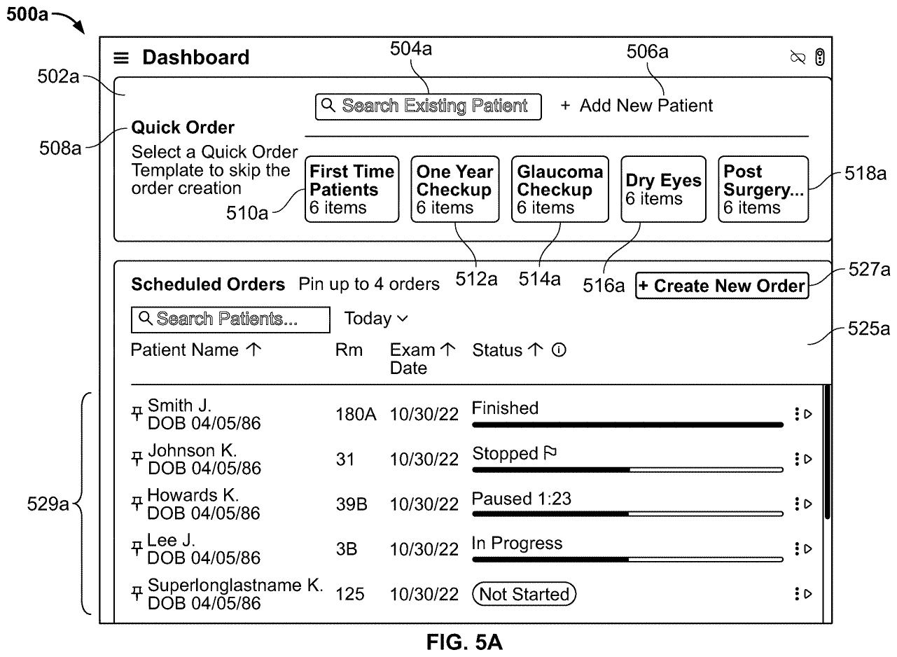

FIG. 4 is a flowchart of a plurality of exemplary steps of a method 400 of delivering an eye exam order to a patient, in accordance with some embodiments of the present specification. In embodiments, the VAD App implements the method 400 on the at least one first computing device 310 in data communication with the head-mounted vision device 305. In various embodiments, the graphical user interfaces generated during the execution of any of the methods described herein may be available for display on a patient device or tablet (at least one first computing device 310), on the head-mounted device, on the clinician's computing device (at least one second computing device 330) or any combination thereof.

In some embodiments, the at least one first computing device 310 serves as both the patient device and clinician device. In some embodiments, the clinician and patient toggle between use of the computing device. In some embodiments, the patient and clinician have their own respective devices. It should be noted herein that the functionalities and graphical user interfaces described throughout this specification are configured such that they allow for the computing device to be used by both patient and clinician or for each of the patient and clinician to have their own dedicated computing devices. Further, a plurality of patient devices may be configured to communicate with one clinician device. Still further, a plurality of patient devices may be configured to communicate with a plurality of clinician devices. In the examples below, the clinician is a primary user of at least one first computing device 310 while the patient is the primary user of head-mounted device 305.

Referring now to FIGS. 3, 4 and 5A through 5F simultaneously, at step 402, the VAD App generates data indicative of a first graphical user interface (GUI) 500a to a) enable a clinical staff person to input information related to one or more patients, input one or more eye exam orders for the one or more patients, and/or review a status of scheduled orders for the one or more patients, and b) enable a patient to self-check-in for an eye exam related to her scheduled order.

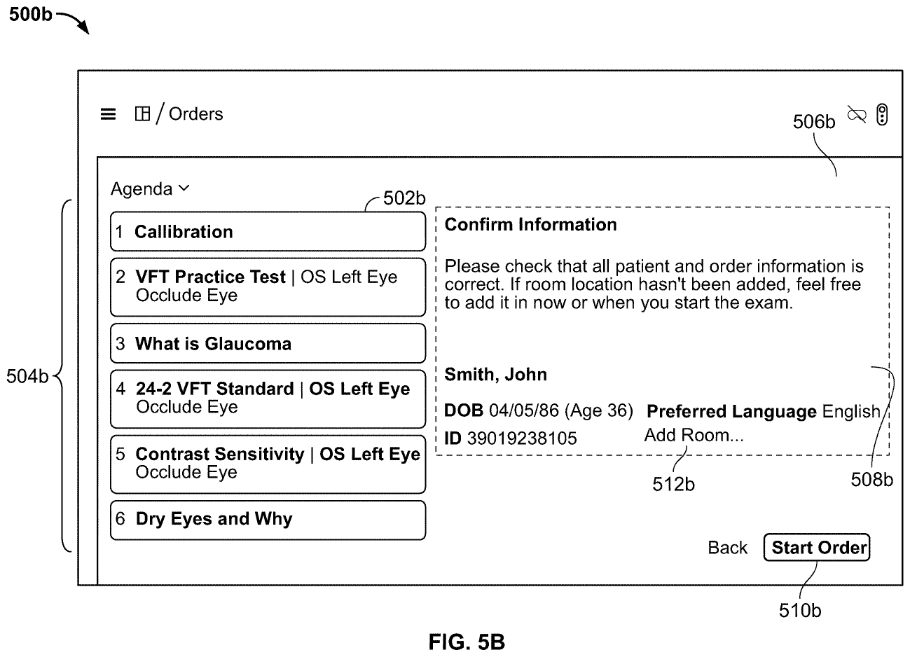

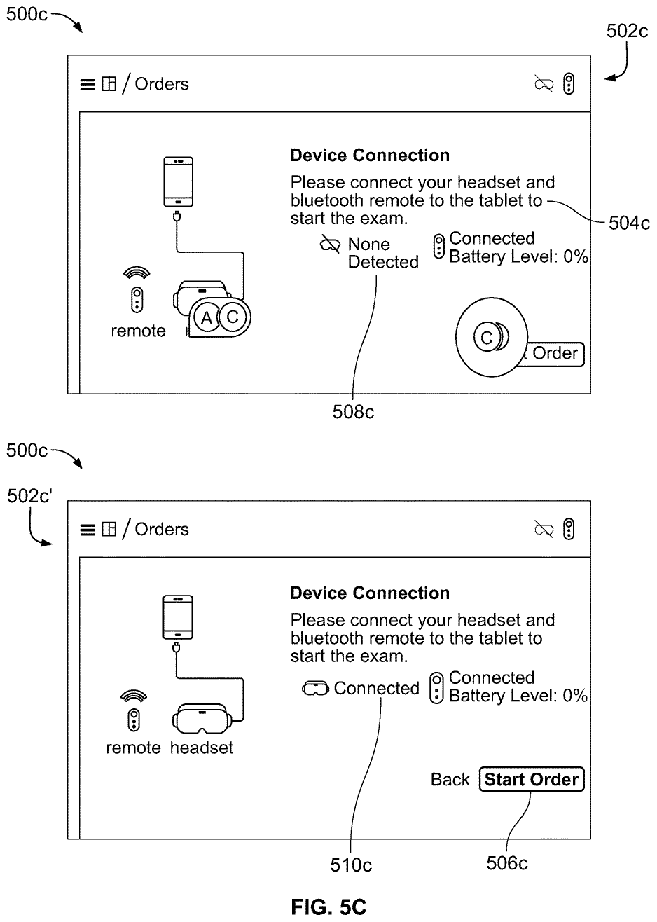

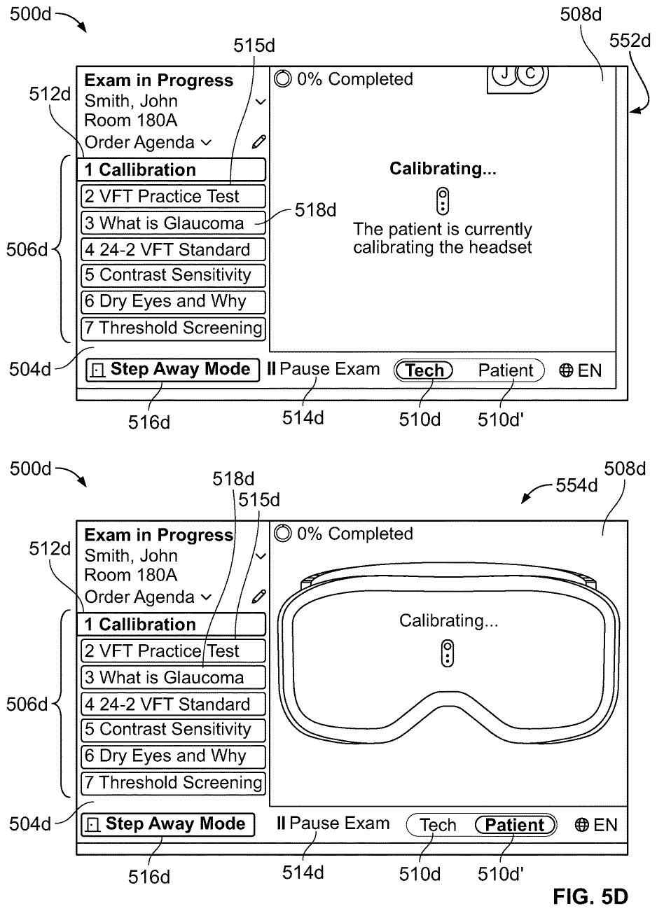

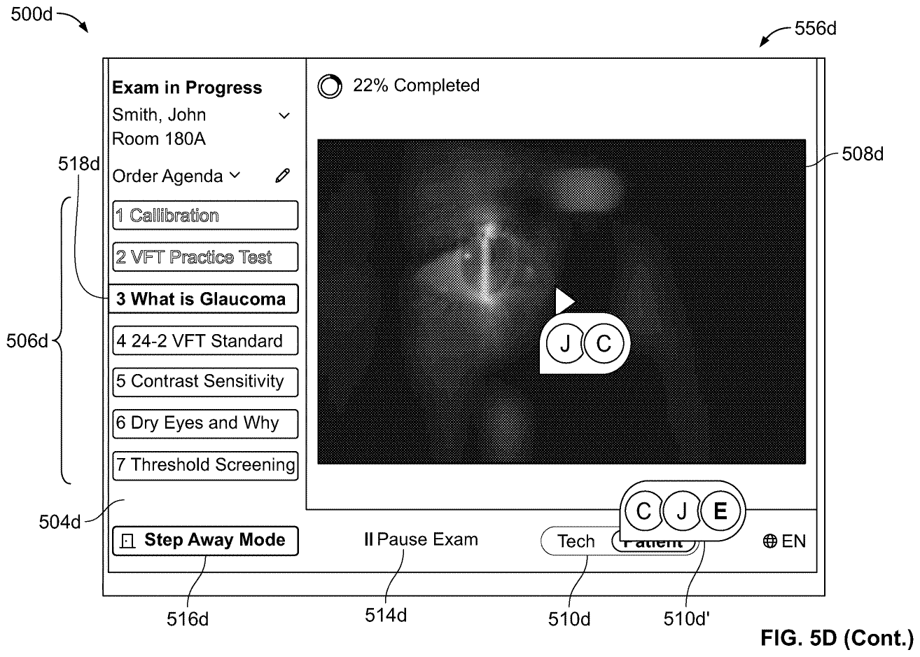

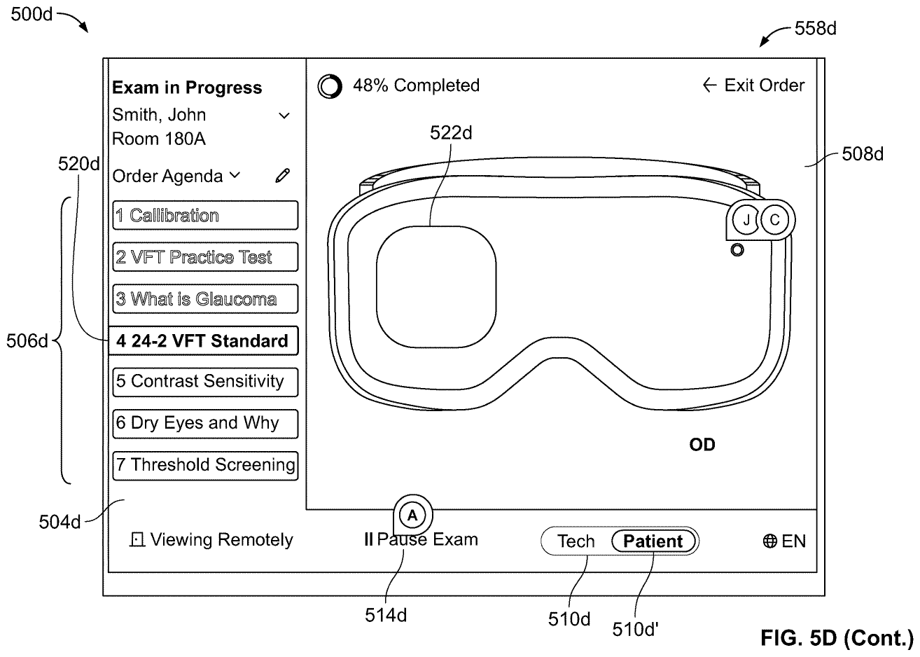

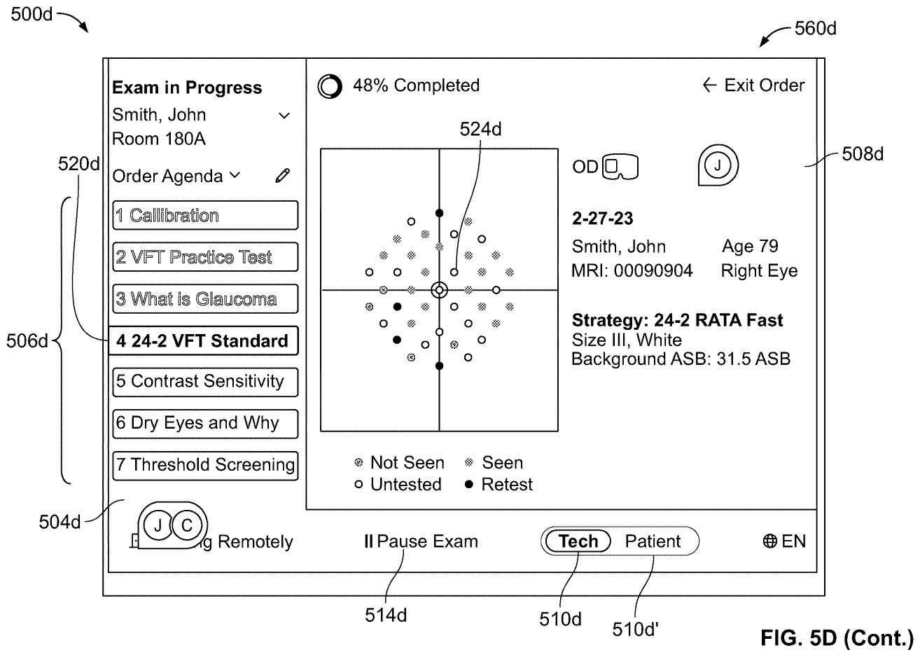

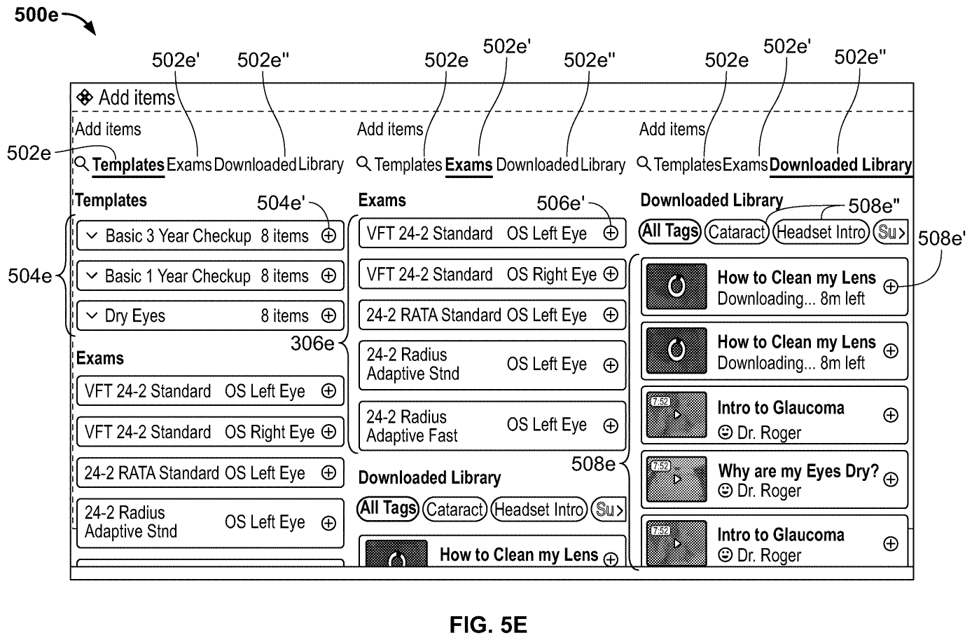

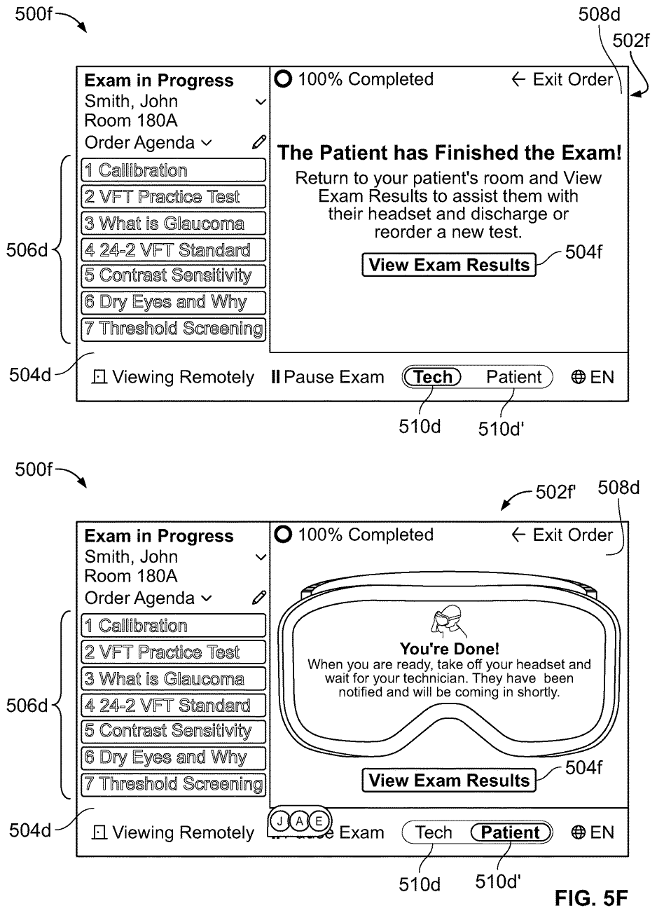

FIG. 5A shows the first GUI 500a generated by the VAD App, in accordance with some embodiments of the present specification. The first GUI 500a is configured as a dashboard that enables a clinical staff person to perform a plurality of tasks while also enabling a patient to self-check-in for an eye exam related to her scheduled order. As shown, the first GUI 500a has a first portion or area 502a including a text box 504a to allow searching information and eye exam orders related to an existing patient. A visual graphical element 506a (titled “add new patient”) when actuated (for example, by touch-based clicking) allows inputting information related to a new patient which is then added to the database 325. Another visual graphical element 508a (titled “quick order”) when actuated allows selecting an order template, from a plurality of pre-stored or pre-configured templates related to a plurality of eye ailments and diagnostics, for a patient. The selected order template is then automatically stored in the database 325 in association with the patient (such as, for example, in association with a unique ID of the patient in the system).

In some embodiments, the first portion or area 502a includes additional visual graphical elements such as a visual graphical element 510a which when actuated allows viewing information related to first time patients, another visual graphical element 512a which when actuated allows viewing related to patients characterized by one year checkup, another visual graphical element 514a which when actuated allows viewing information of patients and orders related to glaucoma ailments, another visual graphical element 516a which when actuated allows viewing information of patients and orders related to dry eye ailments, and yet another visual graphical element 518a which when actuated allows viewing information of patients and orders related to post surgery exams.

A second portion or area 525a allows viewing of a plurality of stored scheduled orders for patients and/or for creating a new eye exam order for a patient. A visual graphical element 527a (titled “create new order”) when actuated allows creation of a new eye exam order for a patient based on default eye exam order fields, patterns, and attributes corresponding to an eye exam. Thus, an order can be created and saved in the system by either selecting a built-in order template (using the visual graphical element 508a) or by actuating the visual graphical element 527a. As a non-limiting example, the second portion or area 525a shows a list 529a of stored scheduled orders for a day along with a plurality of attributes (associated with each of the scheduled orders) such as, for example, patient name, exam room, eye exam date and status.

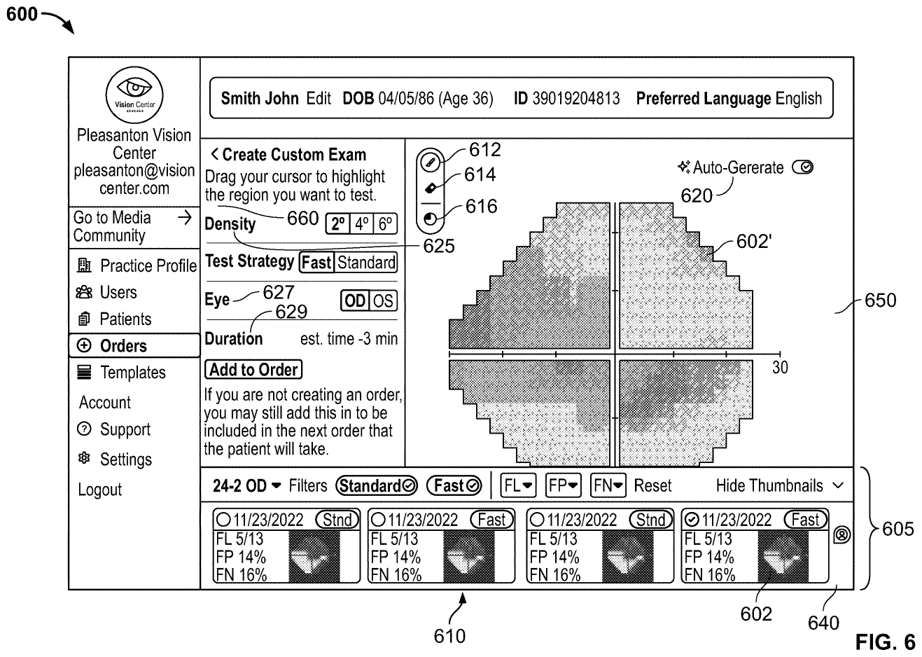

In some embodiments, the VAD App is configured to enable a clinical staff person to create a custom eye exam order for a patient and to give the clinical staff person control of regions for testing and clustering. FIG. 6 shows a GUI 600 generated and presented by the VAD App for enabling creation of a custom eye exam order for a patient, in accordance with some embodiments of the present specification. It should be appreciated that an objective of the GUI 600 is to empower a clinician to custom design (or select a designed eye exam from an auto-populated list) eye exam for a patient subject to certain constraints such as, for example, billable requirements, maximum test time or duration, and historical and/or real-time patient-specific eye exam results. In embodiments, the “billable” requirements refer to Current Procedural Terminology (“CPT”) codes. More specifically, CPT code 92083 (a medical procedure code) specifies a set of conditions that a visual field test must satisfy in order for it to be “billable”. The key requirements (which may be subject to change by the governing authority) are as follows:
i) It is a “threshold test”, which means that “thresholds” or sensitivity is estimated. For example, for an automated static perimetry test, the intensity of the stimulus at which the observer detects the stimulus 50% of the time is estimated.
ii) The visual field test covers three isopters. An isopter refers to a contour of similar sensitivity. Typical visual field test patterns have test points that cover at least three contours of sensitivity values, with one within the other (similar to a topographical map).

In embodiments, the clinical staff person may select a custom region to test based on a previous visual field test as the previous visual field test may help guide which regions to test with greater density. The results of the custom test and the previous visual field might, in some cases, be merged afterwards (as explained in various parts of the specification) to provide a map with a greater density of test points in regions of greater interest, which are typically the boundaries of scotomas. In an alternative embodiment, testing is dynamic—that is, the test may begin with a known test pattern (such as 24-2) but as the testing progresses, the algorithm is configured and designed to determine whether more testing is needed in certain regions and whether less testing is needed in other regions.

In some embodiments, the clinical staff person may choose a previously administered/conducted or a real-time eye exam (based on a predefined standard eye exam), such as eye exam 602, from a horizontal tray 605 configured to display a plurality of eye exams 610 displayed in visual miniature forms/icons. The tray 605 is positioned at a bottom portion 640 of the GUI 600. Selecting the eye exam 602 causes data 602′ indicative of the eye exam 602 to be displayed in a first window 650 positioned above the tray 605.

Subsequently, in some embodiments, the clinical staff person may use at least one of a plurality of drawing tools to define and/or manipulate areas of interest within the displayed data 602′. In embodiments, the plurality of drawing tools includes a first visual graphical element 612 indicative of a brush tool, a second visual graphical element 614 indicative of an eraser tool, and a third visual graphical element 616 indicative of a quadrant or lasso tool. Each of the plurality of drawing tools is configured to enable the clinical staff person to define and/or manipulate one or more test points or locations within the displayed data 602′. The defined and/or manipulated one or more test points or locations are visually highlighted or painted within the displayed data 602′. For example, for a patient already having hemi-vision loss (hemianopia), the clinical staff person can choose to test only the portions (hemi-region) where the patient still has vision in order to reduce test time.

Alternatively, in some embodiments, the clinical staff person may actuate a fourth visual graphical element 620 (titled ‘auto-generate’) which, when actuated, is configured to cause the VAD App to automatically generate a recommended custom eye exam, based on the selected eye exam 602, that may increase test points or locations in areas indicative of poor test results and/or may decrease test points or locations in areas indicative of normal visual field. The auto-generate functionality is configured such that it defines or paints the area(s), within the displayed data 602′, that should be tested more deeply (or more quickly). In some embodiments, the clinical staff person may further manipulate the area(s) (defined by the auto-generate functionality) using the plurality of drawing tools.

Referring back to FIG. 6, a second window 660, positioned above the tray 605 and adjacent the first window 650, displays a fifth visual graphical element 625 indicative of a density of test points or locations in areas of interest or concern (and that toggles among, for example, three density options for selection), a sixth visual graphical element 627 indicative of a left or right eye (and that toggles between left or right eye options for selection) and a seventh visual graphical element 629 indicative of a test time or duration of the custom eye exam, automatically estimated by the VAD App, based on the density option chosen. Thus, in embodiments, density affects test time or duration—when a higher density option is chosen for an area of interest or concern the VAD App provides an estimated higher test time or duration compared to an estimated lower test time or duration when a lower density option is chosen.

As described earlier, new or custom test points or locations are defined as a result of the activation of the auto-generate functionality (by actuating the fourth visual graphical element 620) and/or as a result of the clinical staff person using the plurality of drawing tools within the displayed data 602′. In some embodiments, creation of the new or custom test points or locations (of a custom eye exam) are subject to a plurality of filters or constraints. In some embodiments, the plurality of filters or constraints include billable requirements (that is, for example, must have three isopters and must be billable, as described above), maximum test time or duration allowed, and historical and/or real-time patient-specific eye exam results. In some embodiments, the VAD App is configured to automatically “gray-out” certain selections if choosing them will violate one or more filters or constraints (such as, for example, if selecting a density option will cause the test time or duration to be too large). In some embodiments, the options for modifying test time may include density (for example 2-degree, 4-degree, 6-degree density grids) and/or test strategy (for example, standard or fast) once a custom region is either manually selected (using the brush-like tool) or automatically selected using an algorithm.

In some embodiments, new or custom test points or locations are defined and/or manipulated based on data indicative of a real-time eye exam—that is, areas around good points or locations (where vision is not impaired) will be less densely tested than areas around points or locations where vision is impaired or visual defects are identified.

At step 404, the VAD App enables a patient to check-in using the first GUI 500a. In accordance with an objective of the present specification, the VAD App allows the patient to independently self-check-in using the at least one first computing device 310. In some embodiments, to do so, the patient clicks on his name from the list 529a of scheduled orders appearing for the day in the first GUI 500a. It should be appreciated that enabling the patient to self-check-in and initiate the exam order is desirable since clinicians need to be efficient and service as many patients as possible with the least amount of staff personnel.

At step 406, subsequent to the patient's check-in, the clinical staff person attaches the head-mounted vision device 305 to the patient's head while, concurrently, the VAD App generates data indicative of a second GUI 500b to enable the clinical staff person to initiate a scheduled eye exam order for the patient using the at least one first computing device 310. In some embodiments, attaching the head-mounted vision device 305 to the patient's head automatically places or configures the at least one first computing device 310 into a mode of delivering the eye exam order.

FIG. 5B shows the second GUI 500b generated by the VAD App, in accordance with some embodiments of the present specification. As shown, the second GUI 500b includes a first portion or area 502b that lists a predefined sequential series of content or agenda items 504b related to the eye exam order scheduled for the patient. A second portion or area 506b prompts the patient to check for his personal and order information provided in a third portion or area 508b. The third portion or area 508b also includes a link 512b to enable the patient to select a room in the clinic, if not already selected. A visual graphical element 510b (titled “start order”) when actuated causes the patient's eye exam order to be initiated.

At step 408, the VAD App generates data indicative of a third GUI 500c displaying if a remote device, such as the at least one second computing device 330, and the head-mounted vision assist are activated and in data communication with the at least one first computing device 310.

FIG. 5C shows first and second views 502c, 502c′ of the third GUI 500c generated by the VAD App, in accordance with some embodiments of the present specification. The first view 502c shows the VAD App has sensed that at least one of the head-mounted vision device 305 or the remote device is not connected to the at least one first computing device 310 and therefore displays a message 504c prompting to enable the connection while also displaying a visual graphical element 508c indicating that at least one of the head-mounted vision device 305 or the remote device is not connected to the at least one first computing device 310. The second view 502c′ shows the VAD App sensing that the head-mounted vision device 305 and the remote device are in data communication with the at least one first computing device 310 and hence displays a visual graphical element 510c indicating that the head-mounted vision device 305 and the remote device are in data communication with the at least one first computing device 310. Also, a visual graphical element 506c (titled “start order”) is enabled for actuation in order to initiate the scheduled eye exam order for the patient.

At step 410, the VAD App begins providing, sequentially, one or more content or agenda associated with the eye exam order to the patient through the head-mounted vision device 305 by concurrently generating data indicative of a fourth GUI 500d that has a plurality of views some of which are accessible to a technician or clinical staff person on the at least one first computing device 310 whereas others are accessible to the patient for viewing through the head-mounted vision device 305. In some embodiments, the eye exam order has a predefined sequential series of content or agenda (to be provided to the patient) corresponding to the patient's eye ailment and, hence, the eye exam, diagnostic or test.

FIG. 5D shows first view 552d, second view 554 d, third view 556 d, fourth view 558 d, and fifth view 560d of the fourth GUI 500d generated by the VAD App, in accordance with some embodiments of the present specification. As shown in FIG. 5D, each of the first view 552d, second view 554d, third view 556d, fourth view 558d, and fifth view 560d, has a first portion or area 504d that includes a menu 506d of the predefined sequential series of content or agenda corresponding to the patient's eye ailment and, hence, the eye exam, diagnostic or test. The first portion or area 504d also displays the patient's name.

Each of the first view 552d, second view 554 d, third view 556 d, fourth view 558 d, and fifth view 560d has a second portion or area 508d that includes a toggle-enabled visual graphical element having a first toggle position 510d and a second toggle position 510d′. Actuating the first toggle position 510 d causes each of the first view 552d, second view 554 d, third view 556 d, fourth view 558d, and fifth view 560d to be configured for viewing by the technician or clinical staff person on the at least first computing device 310. Actuating the second toggle position 510d′ causes each of the first view 552d, second view 554 d, third view 556 d, fourth view 558 d, and fifth view 560 d to be configured for viewing by the patient on the head-mounted vision device 305. In some embodiments, the first and second toggle positions 510d, 510d′ are both enabled for actuating by the technician or clinical staff person. In some embodiments, the first toggle position 510d is disabled (for actuating) for the patient. In some embodiments, the second toggle position 510d′ is disabled (for actuating) for the technician or clinical staff person. It should be appreciated that, in some embodiments, the technician or clinical staff person is enabled to view a status of progress of the predefined sequential series of content or agenda by actuating the first toggle position 510d.

In some embodiments, the second portion or area 508d displays data or content corresponding to an agenda, from the menu 506d, that is currently in progress for the patient. The first view 552d displays the data or content in the context of or relevant to the technician or clinical staff person whereas the second view 554d displays the data or content in the context of or relevant to the patient. For example, for a calibration agenda 512d (from the menu 506d) the first view 552d displays (in the second portion or area 508d) a message that the patient is currently calibrating the head-mounted vision device 305 whereas the second view 554d displays (in the second portion or area 508d) an outline of the head-mounted vision device 305 with calibration in progress. The eye exam can be paused by actuating a visual graphical element 514d. The fourth GUI 500d includes another visual graphical element 516d (titled “step away mode”).

Thus, in some embodiments, as the patient progresses through his eye exam order, first view 552d and second view 554d of the fourth GUI 500d are generated, one for the technician or clinical staff person and the other for the patient. Also, as the patient progresses through the predefined sequential series of content or agenda (from the menu 506d), the first GUI 500a screen is also concurrently updated to reflect the patient's progress status on the eye exam.

As shown, in some embodiments, the predefined sequential series of content or agenda in the menu 506d has the calibration agenda 512d followed by a VFT (visual field test) practice test 515d. Conventionally, patients are not instructed to do a practice test because it takes the technician's or clinical staff person's time. It should be appreciated that, in the context of the present specification, the practice test 515d is important and patients take at least one practice test to become proficient at identifying and tracking stimuli related to various eye exams. Practice tests tend to have a great deal of noise or skewed data or sensitivity. The methods and systems of the present specification enable such a practice test to be delivered since the method 400 is patient driven.

In some embodiments, the predefined sequential series of content or agenda includes one or more educational content including text, audio and/or video. For example, as shown in the fourth GUI 500d, the menu 506d includes an education video agenda 518d (for example, “what is glaucoma?”). When the educational video agenda 518d commences for the patient, the second portion or area 508d displays data or content corresponding to the educational video agenda 518d, as shown in the third view 556d (with the first toggle position 510d disabled and the second toggle position 510d′ enabled), of FIG. 5D, of the fourth GUI 500d.

In some embodiments, the predefined sequential series of content or agenda includes at least one eye exam. For example, as shown in fourth and fifth views 558d, 560d, respectively, of the fourth GUI 500d, the menu 506d includes an eye exam agenda 520d (for example, “24-2 VFT Standard”). When the eye exam agenda 520d commences for the patient, the second portion or area 508d displays data or content corresponding to the eye exam agenda 520d, as shown in the fourth and fifth views 558d, 560d, respectively, of FIG. 5D, of the fourth GUI 500d. The fourth view 558d displays data or content accessible to the patient on the head-mounted vision device 305 (with the first toggle position 510d disabled and the second toggle position 510d′ enabled) whereas the fifth view 560d displays data or content accessible to the technician or clinical staff person on the at least one first computing device 310 (with the first toggle position 310d enabled and the second toggle position 510d′ disabled). As a non-limiting example, for the patient, the fourth view 558d displays an outline of the head-mounted vision device 305 with a stimulus being presented in the left eye window 522d. On the other hand, for the technician or clinical staff person, the fifth view 560d displays a battery of stimuli 524d that the patient has not seen, seen, untested, or other characteristics.

In various embodiments, the VAD App also enables the technician or clinical staff person to select and add one or more of a plurality of predefined sequential series of content or agenda to a patient's eye exam order. To enable this, the VAD App, in some embodiments, generates data indicative of a fifth GUI 500e, as shown in FIG. 5E, that is available to the technician or clinical staff person for access on the at least one first computing device 310.

FIG. 5E shows the fifth GUI 500e generated by the VAD App, in accordance with some embodiments of the present specification. As shown, the fifth GUI 500e includes first visual graphical element 502e, second visual graphical element 502e′, and third visual graphical element 502e″ for viewing and selecting/adding eye exam templates, eye exams and educational content, respectively, to the patient's eye exam order. Actuating the first visual graphical element 502e shows a first menu 504e of a plurality of eye exam templates. Each of the plurality of eye exam templates has an associated visual graphical element 504e′ (shown, for example, as a “+” indicator) which when actuated enables selecting and adding the corresponding eye exam template to the patient's eye exam order.

Actuating the second visual graphical element 502e′ shows a second menu 506e of a plurality of eye exams. Each of the plurality of eye exams has an associated visual graphical element 506e′ (shown, for example, as a “+” indicator) which when actuated enables selecting and adding the corresponding eye exam to the patient's eye exam order.

Actuating the third visual graphical element 502e″ shows a third menu 508e of a plurality of educational content. Each of the plurality of educational content has an associated visual graphical element 508e′ (shown, for example, as a “+” indicator) which when actuated enables selecting and adding the corresponding educational content to the patient's eye exam order. In some embodiments, the plurality of educational content may be grouped into one or more categories 508e″ for ease of access by the technician or clinical staff person.

In various embodiments, any subset of a plurality of eye exams, tests or diagnostics may be available for adding as agenda to a patient's eye exam order. The plurality of eye exams, tests or diagnostics may include tests such as, but not limited to, visual field tests (such as confrontation visual field tests, automated static perimetry tests, kinetic visual field tests, ERG, Amsler grid), visual acuity test (certain), visual refraction eye test, color blindness test, and cornea topography. In embodiments, the methods of the present specification focus on, but are not limited to automated static perimetry testing.

At step 412, after the patient has progressed through and completed the predefined sequential series of content or agenda in the eye exam order, the VAD App conveys completion of the eye exam order by generating data indicative of a sixth GUI 500f, as shown in FIG. 5F, that has a first view 502f accessible to the technician or clinical staff person on the at least one first computing device 310 and has a second view 502f′ accessible to the patient for viewing through the head-mounted vision device 305.

FIG. 5F shows the first and second views 502f, 502f′, respectively, of the sixth GUI 500f generated by the VAD App, in accordance with some embodiments of the present specification. Each of the first and second views 502f and 502f′ has the first portion or area 504d that includes the menu 506d of the predefined sequential series of content or agenda corresponding to the patient's eye ailment and, hence, the eye exam, diagnostic or test. Also, each of the first and second views 502fand 502f′ has the second portion or area 508d that includes the toggle-enabled visual graphical element having the first and second toggle positions 510d, 510d′, respectively.

In a non-limiting example, the first view 502f displays a message to the technician or clinical staff person (with the first toggle position 510d enabled) that the patient has completed the ordered eye exam. There may be other messages or prompts such as, for example, “return to your patient's room and view exam results to assist them with their headset and discharge or reorder a new test”. Additionally, a visual graphical element 504f is displayed which when actuated enables the technician or clinical staff person to view the results of the patient's eye exam.

The second view 502f′ displays a message to the patient (with the second toggle position 510d′ enabled) that the eye exam is complete. There may be other messages of prompts such as, for example, “When you are ready, take off your headset and wait for your technician. They have been notified and will be coming in shortly”. Additionally, the visual graphical element 504f is displayed which when actuated enables the patient to view the results of his eye exam.

The above examples are merely illustrative of the many applications of the systems and methods of present specification. Although only a few embodiments of the present invention have been described herein, it should be understood that the present invention might be embodied in many other specific forms without departing from the spirit or scope of the invention. Therefore, the present examples and embodiments are to be considered as illustrative and not restrictive, and the invention may be modified within the scope of the appended claims.

What is claimed is:

1. A computer-implemented method for performing an assessment of a patient's vision,
comprising:
providing a head-mounted vision device configured to be positioned on the patient's head, wherein the head-mounted vision device comprises a display;
providing a first computing device in data communication with the head-mounted vision device;
providing at least one server in data communication with at least one of the first computing device and the head-mounted vision device;
providing a non-transient computer readable medium that is in data communication with at least one of the at least one server and the first computing device, wherein the non-transient computer readable medium comprises a plurality of programmatic instructions that, when executed by at least one processor in at least one of the first computing device and the at least one server:
presenting a series of visual field tests to the patient over a period of time, wherein each of the visual field tests is defined by a visual field test pattern having a plurality of test locations in a visual field of the patient;
receiving visual field test data for each of location of the plurality of test locations;
applying a function to the visual field test data in order to determine a functional relationship between visual field test data at a given one of the plurality of test locations across the series of visual field tests conducted over the period of time;
for each of the plurality of test locations, using the functional relationship to determine a value indicative of a change of sensitivity in the patient's visual field at each of the plurality of test locations;
permuting the series of visual field test data at some or all of the plurality of test locations to generate a unique number of permuted sets of test data;
using the permuted sets of test data, generating a plurality of additional values for each of the plurality of test locations;
generating a distribution of the plurality of additional values for each of the plurality of test locations; and
determining if the change of sensitivity at a given one of the plurality of test locations is significant based on the distribution.

2. The computer-implemented method of claim 1, wherein the function applied to the series of test data is a linear regression function.

3. The computer-implemented method of claim 2, wherein the functional relationship between visual field test data at said given one of the plurality of test locations is defined by a linear regression line.

4. The computer-implemented method of claim 3, further comprising obtaining said linear regression line for every one of the plurality of test locations across said series of test data.

5. The computer-implemented method of claim 4, wherein using the functional relationship to determine the value indicative of the change of sensitivity in the patient's visual field comprises calculating a slope of the linear regression line for each of the plurality of test locations, wherein the slope is indicative of a rate of change of sensitivity at each of the plurality of test locations.

6. The computer-implemented method of claim 1, wherein using the permuted sets of test data to generate the plurality of additional values comprises generating a plurality of slope values for each of the plurality of test locations.

7. The computer-implemented method of claim 6, wherein said generation of the plurality of slope values for each of the plurality of test locations comprises fitting a line through each permuted test data by applying linear regression to the permuted test data and calculating a slope of the linear regression line for either the unique number of permuted test data or for a predetermined number of randomly chosen permutations from the unique number of permuted test data.

8. The computer-implemented method of claim 1, wherein the visual field test data are indicative of a degree of sensitivity at a given one of the plurality of test locations in the patient's visual field.

9. The computer-implemented method of claim 1, wherein the series of visual field test data includes N data, and wherein the number of permuted sets of test data is N! (N factorial).

10. The computer-implemented method of claim 1, wherein the distribution is indicative of an effect of random noise on the series of visual field test data at a given one of the plurality of test locations.

11. The computer-implemented method of claim 1, wherein said significance of the change of sensitivity at the given one of the plurality of test locations is determined based on whether both a first condition and a second condition are true.

12. The computer-implemented method of claim 11, wherein the first condition is indicative of whether the change of sensitivity at the given one of the plurality of test locations is worse than a first predefined threshold.

13. The computer-implemented method of claim 12, wherein the first predefined threshold is within a range of −0.3 dB/year to −1.5 dB/year.

14. The computer-implemented method of claim 11, wherein the second condition is indicative of whether the change of sensitivity at the given one of the plurality of test locations is lower than a second predefined threshold of the distribution of the plurality of additional values for each of the plurality of test locations.

15. The computer-implemented method of claim 11, wherein the plurality of additional values comprises a plurality of slope values for each of the plurality of test locations.

16. The computer-implemented method of claim 1, further comprising causing at least one graphical user interface to be generated in the display of the head-mounted vision device or in the first computing device, wherein the graphical user interface is configured to display the change of sensitivity at a given one of the plurality of the test locations.

17. The computer-implemented method of claim 16, wherein the graphical user interface is further configured to visually highlight whether the change of sensitivity at the given one of the plurality of the test locations is significant.

18. The computer-implemented method of claim 17, wherein the graphical user interface is further configured to display a message indicative of a total number of the plurality of the test locations determined to have significant changes.

19. The computer-implemented method of claim 18, wherein the change of sensitivity is a rate of change of sensitivity.
